(19) United States
(12) Patent Application Publication
(10) Pub. No.: US 2020/0000516 A1
(43) Pub. Date: Jan. 2, 2020

(54) STERILE DISPOSABLE BIPOLAR ABLATION NEEDLE, ASSOCIATED SYSTEM, AND METHOD OF USE

(71) Applicant: Daniel Igor Branovan, Livingston, NJ (US)

(72) Inventor: Daniel Igor Branovan, Livingston, NJ (US)

(21) Appl. No.: 16/258,619

(22) Filed: Jan. 27, 2019

Related U.S. Application Data
(63) Continuation-in-part of application No. 15/188,952, filed on Jun. 21, 2016, now abandoned.

(51) Int. Cl. A61B 18/14

(52) U.S. Cl. CPC A61B 18/1477; A61B 2018/00577

(57) ABSTRACT

A sterile disposable bipolar ablation needle (the needle) comprising an exposed outer electrode and an inner electrode is described. The needle can also include an insulating layer disposed between the inner electrode and the outer electrode. The needle can further include a first exposed region of the needle disposed near a proximate end of the needle, the first exposed region including a first exposed portion of the inner electrode and a first exposed portion of the outer electrode, the first exposed region constructed to provide contact to a connector, and a second exposed region of the needle disposed near a distal end of the needle. A stylet can be provided for use with the needle and a sterile cover can be provided to cover the needle and maintain sterility of the needle and the stylet.

### CROSS REFERENCE TO RELATED APPLICATIONS

This application is a continuation-in-part of application Ser. No. 15/188,952, entitle “Disposable Bipolar Coaxial Radio Frequency Ablation Needle, System and Method,” and filed on Jun. 21, 2016, the entire contents of which are incorporated herein by reference.

### FIELD

Embodiments relate generally to electrical surgical (or electrosurgery) instruments, and, more particularly, to sterile disposable bipolar ablation instruments (e.g., needles), and systems and methods of using the same.

### BACKGROUND

A common treatment for malignant tumors in human organs, such as nodules on the thyroid or renal masses on the kidney, is surgical removal of most of the respective organ tissue. For example, a thyroidectomy may be performed to deal with malignant thyroid tumors, a procedure which unfortunately results in removal of most of the thyroid tissue. Moreover, undergoing thyroid surgery often poses risks, such as nerve damage or damage to parathyroid glands, and may require that the patient take thyroid hormone supplements following surgery. Alternatives to thyroidectomy are known in the art for non-malignant tumors, including radio frequency (RF) ablation techniques in which the temperature of the target tissue may be raised to a temperature of 60° C. or higher.

For example, Holmer et al. have evaluated ablation methods, as reported in “Bipolar Radiofrequency Ablation for Nodular Thyroid Disease—ex Vivo and in Vivo Evaluation of a Dose-Response Relationship,” J. Surg. Res. 2009 Oct. 29. A study in percutaneous RF ablation for benign thyroid nodules was also described by Kim et al. in “Radiofrequency Ablation of Benign Cold Thyroid Nodules: Initial Clinical Experience,” Thyroid, 2006 April, 16(4):361-7. Kim et al. reported that the ablation electrode used was internally cooled, and that a majority of the patients required conscious sedation when undergoing the ablation procedure.

While there are known in the art RF devices suitable for use in the ablation of liver tumors, for example, most such devices require an extended period of use of from five to ten minutes per session. This length of time makes it impractical to use a conventional RF device on thyroid nodules, in particular, as the thyroid will move with the swallowing motions of the patient. A need may exist for an electrosurgery, electrocautery, or percutaneous ablation instrument that will allow for relatively quick excision of a malignant tissue or tumor.

The existing methods of treatment by high-frequency ablation (HFA) typically suggest the use of either mono-polar or bipolar method. In varying degrees, each of these techniques has certain limitations defined by the physical characteristics of the ablation instrument.

For example, with a mono-polar method, the patient's electrode must have a constant reliable electrical contact with the patient's body, which in some cases is not always easy to ensure or is not always convenient. Meanwhile, high-frequency currents flowing through the patient's body from the patient's electrode to an active electrode typically occur through extensive portions of healthy tissues and may have the additional undesirable effects on those tissues. Further, there could be a potential negative impact of these currents upon, for instance, a pacemaker.

Bipolar ablation requires existence of two electrodes located close to each other to have impact only on the portion of body located between these electrodes. Unlike monopolar ablation, the bipolar method allows to limit the impact of electric current only to the “area of interest”, and also substantially reduce the exposure time to achieve the desired result (coagulation size). In general, a tool for a bipolar ablation is used to work on the visible surface. Alternatively, special tools are used with thin parallel conductive electrodes (needles) creating a possibility of impact upon internal areas, such as patent BY 19188 C1 2015.06.30, used in the treatment of thyroid neoplasms. Nevertheless, the relative bulkiness of the tool, and the presence of two needles with the protruding edge of the insulator on each electrode hinders the penetration and control of their position in the exposure area, increases the size of the wound and may lead to undesirable emotions of the patient.

In addition, the tools mentioned above may be difficult to sterilize, and may not be suitable as a disposable instrument (e.g., for single use).

### SUMMARY

Some implementations can include a sterile disposable bipolar ablation needle comprising an outer conductor and an inner conductor separated by an insulating layer disposed between the inner conductor and the outer conductor, where the outer conductor is exposed and not covered by an insulating layer. The sterile disposable bipolar ablation needle can further include a first exposed region of the sterile disposable bipolar ablation needle disposed near a proximate end of the sterile disposable bipolar ablation needle, the first exposed region including a first exposed portion of the inner electrode and a first exposed portion of the outer electrode, the first exposed region constructed to provide contact to a connector, and a second exposed region of the sterile disposable bipolar ablation needle disposed near a distal end of the sterile disposable bipolar ablation needle, the second exposed region forming the active region of the sterile disposable bipolar ablation needle and including a second exposed portion of the inner conductor and a second exposed portion of the outer conductor.

The sterile disposable bipolar ablation needle can also include a connector coupled to the sterile disposable bipolar ablation needle and having a first contact contacting the first exposed portion of the outer conductor and a second contact contacting the first exposed portion of the inner conductor. The inner conductor and the outer conductor can be formed from stainless steel similar to that used for surgical needles and in a manner similar to that used for surgical needles.

The insulating internal dielectric layer can be formed from medical lacquer or other suitable material. The second exposed region of the inner electrode can be about 7 mm and the second exposed portion of the outer electrode can be about 7 mm.

The first exposed portion can include an exposed portion of the first insulating layer. The second exposed portion can include an exposed portion of the first insulating layer. A diameter of the inner needle can be about 0.5 mm. A diameter of the outer needle can be about 0.9 mm.

Some implementations can include a high frequency ablation system having an electrosurgical generator, and a coaxial bipolar ablation instrument (as described above). The system can also include an adapter cable having a first end constructed to be coupled to the instrument and a second end constructed to connect to the electrosurgical generator, a power supply coupled to the electrosurgical generator; and a control switch coupled to the electrosurgical generator.

The system can also include a connector coupled to the instrument and having a first contact contacting the first exposed portion of the outer needle and a second contact contacting the first exposed portion of the inner needle. The inner needle and the outer needle can be formed from stainless steel surgical needles or needles similar to the needles used for spinal tap and spinal anesthesia.

The insulating layer can be formed from medical lacquer. The second exposed region of the inner needle can be about 7 mm and the second exposed portion of the outer needle can be about 7 mm.

The first exposed portion can include an exposed portion of the first insulating layer. The second exposed portion can include an exposed portion of the first insulating layer.

A diameter of the inner needle can be about 0.5 mm. A diameter of the outer needle can be about 0.9 mm.

Some implementations can include a method for ablating a tissue in a patient comprises the steps of: obtaining an instrument having both a first electrode assembly and a second electrode assembly retained in a handle, the handle retaining the first electrode section in a generally parallel relationship with the second electrode section so as to define a substantially linear bipolar ablation zone between a portion of the first electrode assembly and a portion of the second electrode assembly, the bipolar ablation zone being in an offset and substantially parallel alignment with an axis of the handle; inserting the bipolar ablation zone into a patient proximate a region containing the tissue; determining that a portion of the tissue has been positioned within the bipolar ablation zone; powering the first electrode assembly and the second electrode assembly for a predetermined time period so as to produce a predefined level of ablative RF power in the bipolar ablation zone; and removing the bipolar ablation zone from the patient.

Additional features and advantages of the disclosed subject matter are set forth in the detailed description which follows and/or will be apparent to those skilled in the art.

### BRIEF DESCRIPTION OF THE DRAWINGS

FIG. 1 is a diagram of an example sterile disposable bipolar ablation needle with an aspirating needle and a sterile cover in place in accordance with some implementations.

FIG. 2A is a diagram of an example sterile disposable bipolar ablation needle in accordance with some implementations.

FIG. 2B is a diagram of an example sterile disposable bipolar ablation needle showing details of the electrodes and working area in accordance with some implementations.

FIG. 3 is a diagram of an example stylet for use in connection with a sterile disposable bipolar ablation needle in accordance with some implementations.

FIG. 4 is a diagram of an example sterile cover for a sterile disposable bipolar ablation needle in accordance with some implementations.

FIG. 5 is a diagram showing details of the working area of an example sterile disposable bipolar ablation needle in accordance with some implementations.

FIG. 6 is a diagram showing an example electrical connection to a sterile disposable bipolar ablation needle in accordance with some implementations.

FIG. 7 is a diagram showing an example electrical connection to a sterile disposable bipolar ablation needle in accordance with some implementations.

FIG. 8 is a diagram showing an example electrosurgery environment in accordance with some implementations.

FIG. 9 is a flowchart of an example electrosurgery method in accordance with some implementations.

### DETAILED DESCRIPTION

Some implementations can include a bipolar radio frequency (RF) ablation, electrosurgery, or electrocautery instrument (e.g., a sterile disposable bipolar ablation needle) designed for percutaneous ablation of tissue in a human tissue structure, such as a thyroid nodule or renal mass. The sterile disposable bipolar ablation needle may be inserted through a patient's skin to a thyroid nodule or to renal cell carcinoma under guidance of an imaging system (e.g., ultrasound). Activation of the sterile disposable bipolar ablation needle serves to quickly destroy malignant tissue (e.g., neoplasm).

In some implementations, the out conductor of the sterile disposable bipolar ablation needle uncoated and a sharpening angle of 12° can be used. According to tests conducted by the inventor of the disclosed subject matter, these features make puncturing the skin and thyroid capsule of a patient much easier for a physician. As a result, the entire procedure becomes less painful and more tolerable for a patient.

Moreover, by not requiring a coating on the outer conductor of the sterile disposable bipolar ablation needle, the manufacturing process is made simpler and less expensive and is better suited for mass production.

The configuration of the work zone of the sterile disposable bipolar ablation needle provides for an ability to localize the region of ablation and to thus minimize peripheral damage to surrounding, healthy tissue.

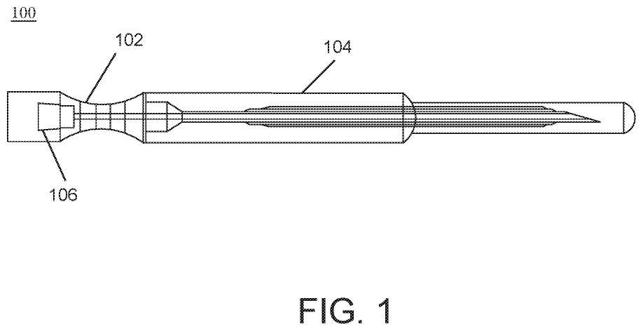

FIG. 1 is a diagram of an example sterile disposable bipolar ablation needle system 100 having a sterile disposable bipolar ablation needle 102, a protective cover 104 to help protect and maintain sterility of the sterile disposable bipolar ablation needle, and a stylet 106.

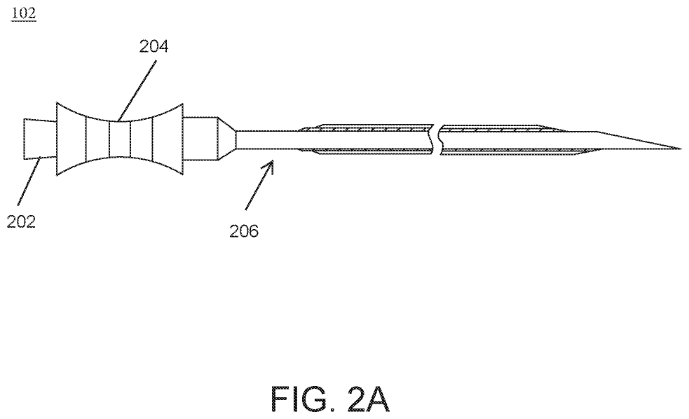

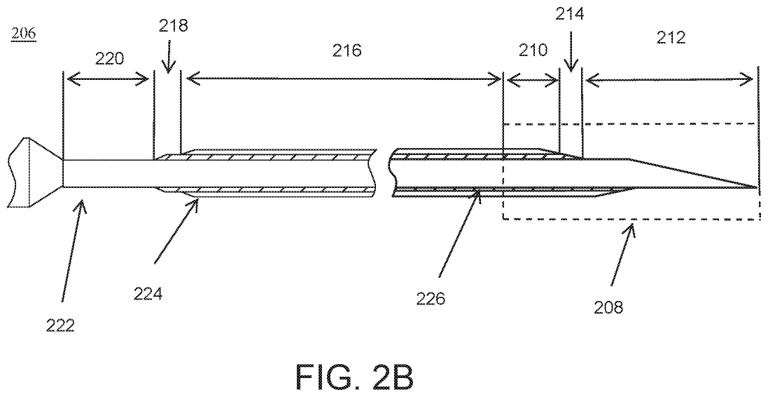

FIG. 2A is a diagram of an example sterile disposable bipolar ablation needle 102, which includes a stylet socket 202, a grip 204, and a needle 206. The stylet socket 202 is a cannula for connecting with a syringe for aspiration.

FIG. 2B is a diagram of an example sterile disposable bipolar ablation needle 102 showing details of the electrodes and working zone. In particular, the sterile disposable bipolar ablation needle 102 includes a working zone 208 that has a portion of the outer electrode 210, an exposed portion of the inner electrode 212, and an exposed portion of the internal dielectric layer 214. The portion of the outer electrode 210 can have a length of about 1 mm-15 mm, the exposed portion of the inner electrode 212 can have a length of about 1 mm-15 mm, and the exposed portion of the dielectric 214 can have a length of about 1 mm-2 mm. In some implementations, an outer maximum diameter of the working area of the tool should be no more than 0.9 mm. The length of the working area can be from about from 3 to 32 mm. Maximum immersion depth (i.e., length from the PCB adapter to the end of the needle) may be no more than 60 mm.

Section 216 can have a length that depends on the length of the working zone and can be about 46 mm to 75 mm. Section 218 can have a length of about 2 mm. Section 220 can have a length of about 10 mm.

The sterile disposable bipolar ablation needle 102 includes an inner electrode 222 that is a needle shaped electrode with a hollow interior and an inner diameter of about 0.5 mm. The sterile disposable bipolar ablation needle 102 also includes an outer electrode 224 having an inner diameter of about 0.9 mm. The sterile disposable bipolar ablation needle 102 also includes an internal dielectric layer 226 disposed between the inner electrode 222 and the outer electrode 224. In some implementations, the dielectric layer 226 can provide an insulation resistance between the external and internal needle (electrodes), R, of at least about 3 kΩ.

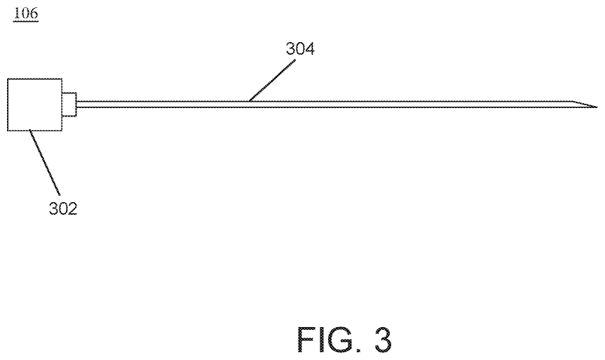

FIG. 3 is a diagram of an example stylet 106 having a stylet connector 302 and a stylet needle portion 306. The stylet 106 can be used during a procedure to ease aspiration of fluid before the ablation procedure. The stylet 106 can be inserted loosely into the hole of the inner needle 102 and has a holder for easy removal from the hole of the needle. The stylet 106 is optional; the sterile disposable bipolar ablation needle 102 can be used by itself. Typically, the stylet 106 is removed before the ablation procedure to aspirate the fluid component of the node. Ablation procedure can be performed also with the presence of the stylet 106.

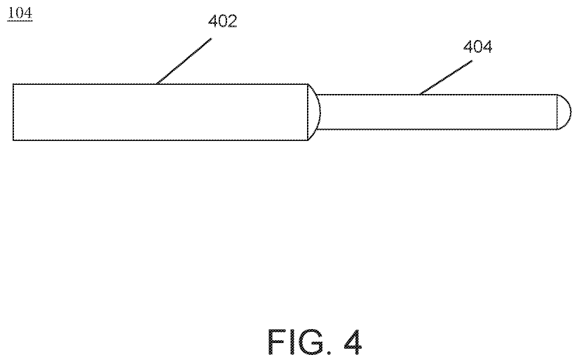

FIG. 4 is a diagram of an example sterile cover 104 having a first cover portion 402 and a second cover portion 404, where the first cover portion 402 has a diameter greater than a diameter of the second cover portion 404.

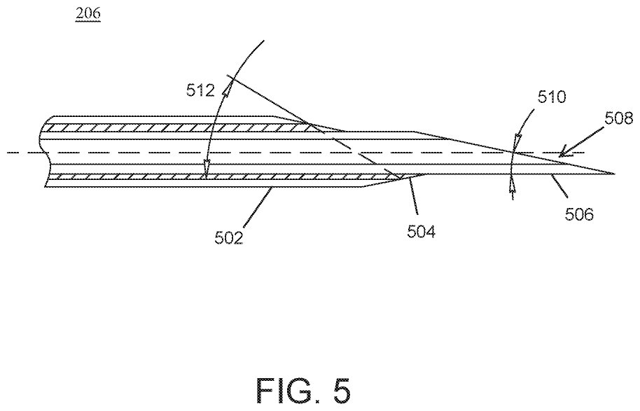

FIG. 5 is a diagram showing details of the working zone 208 of an example sterile disposable bipolar ablation needle 206 in accordance with some implementations. In particular, FIG. 5 shows an outer needle shaped electrode 502 (210 in FIG. 2B), a needle shaped internal dielectric layer 504 (214 in FIG. 2B), and an inner electrode 506 (212 in FIG. 2B). The inner electrode 506 includes a hollow interior space 508 that is constructed to receive a stylet (e.g., 106). The working zone of the sterile disposable bipolar ablation needle 206 includes a first sharpening angle 510 of the inner electrode. The first sharpening angle 510 can be about 12°±2°—for an embodiment with a long cut or 18°±2° for an embodiment with a short cut. The working zone of the sterile disposable bipolar ablation needle 206 includes a second sharpening angle 512 of the inner electrode. The second sharpening angle 512 can be in the range of about 20-90° and can include a chamfer. In some implementations, the maximum diameter of the working area of the tool should be no more than 0.9 mm. In some implementations, the length of the working area can be from about 3 mm to 32 mm.

In some implementations, maximum immersion depth (e.g., length from the PCB adapter to the end of the needle) may be no more than about 60 mm.

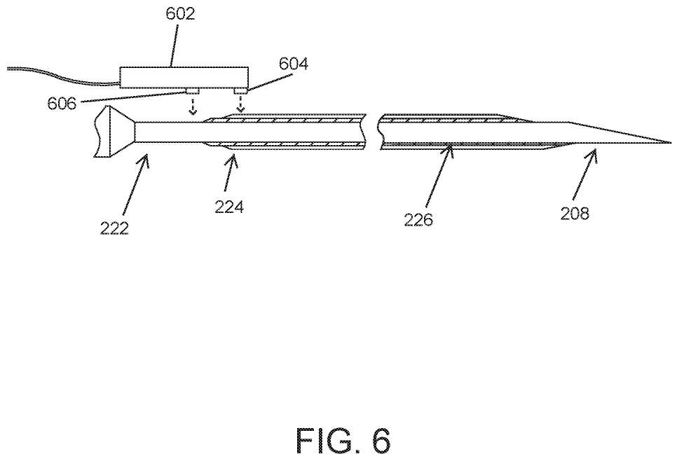

FIG. 6 is a diagram of an example sterile disposable bipolar ablation needle 206. The sterile disposable bipolar ablation needle 206 can include an inner electrode 222, an outer electrode 224, and an internal dielectric (or insulating) layer 226.

The sterile disposable bipolar ablation needle 206 has two exposed contact areas: 1) a first exposed contact area near a proximate end of the sterile disposable bipolar ablation needle 206 where a connector 602 connects to the sterile disposable bipolar ablation needle 206 and to an adapter cable connected to an electrosurgical generator. The connector 602 includes an outer electrode contact 604 and an inner electrode contact 606; and 2) a second exposed contact area on a distal end (e.g., the working zone) that forms the active portion of the instrument where RF energy is emitted into tissue. As described above, the sterile disposable bipolar ablation needle 206 can also include sterile cover 104 to maintain the sterility the sterile disposable bipolar ablation needle 206 while the connector 602 is being attached. The cover 104 can be removed just prior to a surgical procedure.

The inner electrodes and outer electrodes can be stainless steel (e.g., similar to that material and/or shape of spinal tap needles) of the appropriate gauges sized for a given ablation procedure. The insulating material can be a medically safe material, such as Mastersilver made by Renfert (a German company), Mastersilver is a lacquer used in prosthetic dentistry, or other suitable medically safe insulating material.

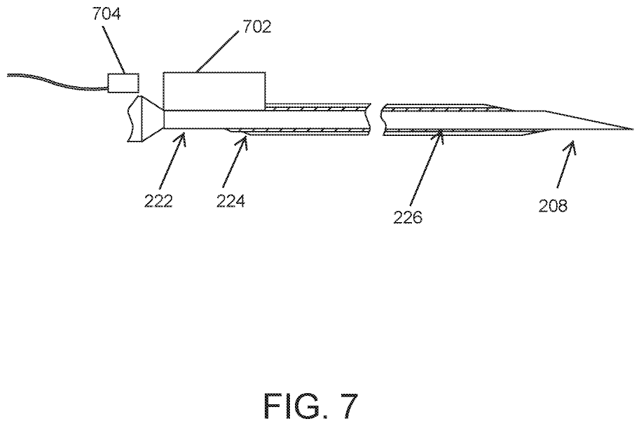

FIG. 7 shows an implementation in which the connector 702 is integrated with the sterile disposable bipolar ablation needle 206 and a connector 704 can be coupled to the connector 702 on the sterile disposable bipolar ablation needle 206. This could increase the cost of the sterile disposable bipolar ablation needle 206, but may have advantages such as having an instrument manufactured with the connector 702 attached at the factory.

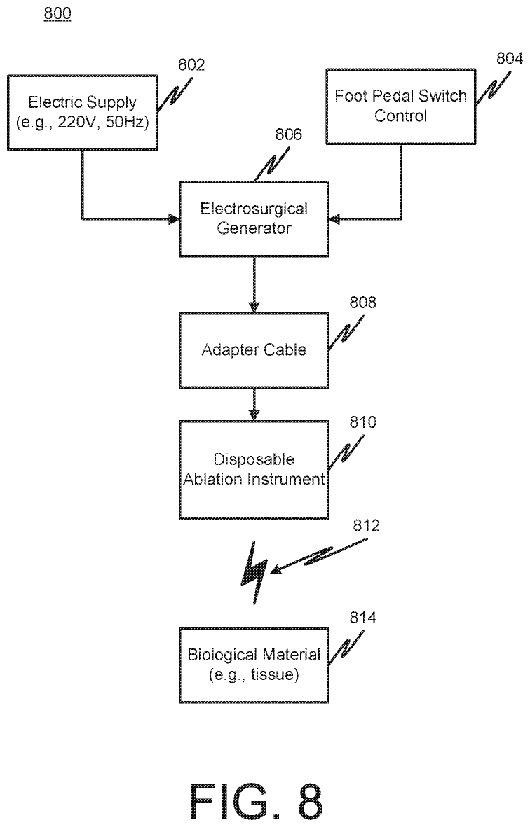

FIG. 8 is a diagrammatic illustration of an example electrosurgery system 800 utilizing a sterile disposable bipolar ablation needle 810 in accordance with some implementations. In particular, the system 800 includes an electric power supply 802 (e.g., a 220V, 50 Hz supply), foot pedal switch control 804, an electrosurgical generator 806, an adapter cable 808, a sterile disposable bipolar ablation needle 810, which can produce energy 812 that impinges on biological material 814 (e.g., tissue).

Some implementations can include a sterile disposable bipolar ablation needle 206 comprising an outer electrode and an inner electrode. The sterile disposable bipolar ablation needle 206 can also include an insulating layer disposed between the inner electrode and the outer electrode. The sterile disposable bipolar ablation needle 206 can further include a first exposed region of the instrument disposed near a proximate end of the sterile disposable bipolar ablation needle 206, the first exposed region including a first exposed portion of the inner electrode and a first exposed portion of the outer electrode, the first exposed region constructed to provide contact to a connector, and a second exposed region of the sterile disposable bipolar ablation needle 206 disposed near a distal end of the sterile disposable bipolar ablation needle 206, the second exposed region forming the active region of the sterile disposable bipolar ablation needle 206 and including a second exposed portion of the inner electrode and a second exposed portion of the outer electrode.

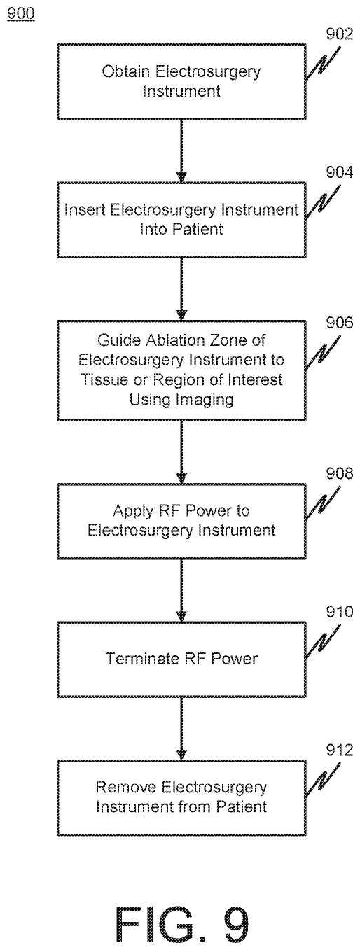

FIG. 9 is a flow diagram illustrating an example method 900 of operation of the sterile disposable bipolar ablation needle of FIGS. 1-7 in accordance with some implementations. The process begins at 902, where a sterile disposable bipolar ablation needle or instrument (e.g., 810) is obtained. If the sterile disposable bipolar ablation needle is not already connected to an electrosurgical generator, then sterile disposable bipolar ablation needle can be attached to an electrosurgical generator via an adapter cable. The process continues to 904.

At 904, the sterile disposable bipolar ablation needle is inserted into a patient. The process continues to 906.

At 906, the insertion of the sterile disposable bipolar ablation needle is optionally guided to a region of interest using an imaging technique such as ultrasound or the like. The process continues to 908.

At 908, RF power is applied to the instrument. The process continues to 910.

At 910, the application of RF power is stopped or terminated. The termination of RF power can occur after a predetermined amount of time or after an amount of time based on the condition of the tissue. The process continues to 912, where the sterile disposable bipolar ablation needle is removed from the patient. The sterile disposable bipolar ablation needle can optionally be discarded.

It is to be understood that the description herein is exemplary only and is intended to provide an overview for the understanding of the nature and character of the disclosed subject matter. The accompanying drawings are included to provide a further understanding of various features and embodiments of the method and apparatus, which, together with their description serve to explain the principles and operation of the disclosed subject matter. Thus, while the disclosed subject matter has been described with reference to particular embodiments or implementations, it will be understood that the disclosed subject matter is not limited to the particular constructions and methods herein described and/or shown in the drawings, but also comprises any modifications or equivalents within the scope of the disclosed subject matter.

It is, therefore, apparent that there is provided, in accordance with the various embodiments disclosed herein, a sterile disposable bipolar ablation needle, and associated system and method.

While the disclosed subject matter has been described in conjunction with a number of embodiments, it is evident that many alternatives, modifications and variations would be or are apparent to those of ordinary skill in the applicable arts. Accordingly, Applicant intends to embrace all such alternatives, modifications, equivalents and variations that are within the spirit and scope of the disclosed subject matter.

What is claimed is:

1. A sterile disposable bipolar ablation needle comprising:
an outer electrode;
an inner electrode disposed within the outer electrode;
an insulating layer disposed between the inner electrode and the outer electrode;
a first exposed region of the sterile disposable bipolar ablation needle disposed near a proximate end of the sterile disposable bipolar ablation needle, the first exposed region including a first exposed portion of the inner electrode and a first exposed portion of the outer electrode, the first exposed region constructed to provide contact to a connector; and
a second exposed region of the sterile disposable bipolar ablation needle disposed near a distal end of the sterile disposable bipolar ablation needle, the second exposed region forming an active region of the sterile disposable bipolar ablation needle and including a second exposed portion of the inner electrode and a second exposed portion of the outer electrode.

2. The sterile disposable bipolar ablation needle of claim 1, further comprising a connector coupled to the sterile disposable bipolar ablation needle and having a first contact contacting the first exposed portion of the outer electrode and a second contact contacting the first exposed portion of the inner electrode.

3. The sterile disposable bipolar ablation needle of claim 1, wherein the inner electrode and the outer electrode are formed from stainless steel.

4. The sterile disposable bipolar ablation needle of claim 1, wherein the insulating layer is formed from medical lacquer.

5. The sterile disposable bipolar ablation needle of claim 1, wherein the second exposed region of the inner electrode is in a range of about 1 mm to 15 mm and the second exposed portion of the outer electrode is in a range of about 1 mm to 15 mm.

6. The sterile disposable bipolar ablation needle of claim 1, wherein the first exposed portion includes an exposed portion of the insulating layer.

7. The sterile disposable bipolar ablation needle of claim 1, wherein the second exposed portion includes an exposed portion of the insulating layer.

8. The sterile disposable bipolar ablation needle of claim 1, wherein an inner diameter of the inner electrode is about 0.5 mm.

9. The sterile disposable bipolar ablation needle of claim 1, wherein a diameter of the outer electrode is about 0.9 mm.

10. The sterile disposable bipolar ablation needle of claim 1, wherein a working zone of the sterile disposable bipolar ablation needle includes a first sharpening angle of the inner electrode, and wherein the first sharpening angle can be about 12°±2° for a long cut or 18°±2° for a short cut.

11. The sterile disposable bipolar ablation needle of claim 10, wherein the working zone of the sterile disposable bipolar ablation needle includes a second sharpening angle of the inner electrode, wherein the second sharpening angle can be in the range of about 20° to 90°.

12. The sterile disposable bipolar ablation needle of claim 11, wherein the second sharpening angle includes a chamfer.

13. The sterile disposable bipolar ablation needle of claim 12, wherein a length of the working area can be within the range of about 3 mm to 32 mm.

14. The sterile disposable bipolar ablation needle of claim 1, wherein a maximum immersion depth is less than or equal to about 60 mm.
